(19) United States
(12) Patent Application Publication
(10) Pub. No.: US 2026/0355078 A1
(43) Pub. Date: Nov. 12, 2026

(54) TOOTH WHITENING COMPOSITION, KIT AND METHODOLOGY

(71) Applicant: DFL INDÚSTRIA E COMÉRCIO S/A, Rio de Janeiro (BR)

(72) Inventors: Joel KOS, Rio de Janeiro (BR); Rafael CISNE DE PAULA, Rio de Janeiro (BR)

(21) Appl. No.: 19/120,103

(22) PCT Filed: Nov. 13, 2023
(86) PCT No.: PCT/BR2023/050388

§ 371 Date: Apr. 10, 2025

(30) Foreign Application Priority Data
Nov. 16, 2022 (BR) 1020220232555

(51) Int. Cl. A61K 8/22; A61Q 11/02

(52) U.S. Cl. CPC A61K 8/22; A61Q 11/02; A61K 2800/48; A61K 2800/5922; A61K 2800/882

(57) ABSTRACT

A two-component tooth whitening composition based on 35% hydrogen peroxide, which provides effective tooth whitening in a safer and more convenient manner. The composition provides the desired effect in a single application step with less penetration of the hydrogen peroxide into the tooth pulp, which significantly reduces tooth sensitivity after whitening. A kit comprising the components of the composition and instructions for preparing the composition by mixing the two components; and an aesthetic method for tooth whitening using the whitening composition.

### FIELD OF INVENTION

The invention involves preparations containing inorganic ingredients used in teeth whitening. The invention's composition in particular comprises hydrogen peroxide and is applicable to dental whitening protocols implemented by dental offices.

### INVENTION BACKGROUND

The dental whitening process comprises a reaction between the molecules in the respective whitening agent and the dental structures, which breaks down the substances that color teeth-called chromophores-into smaller molecules and allow them to become soluble in water. The main agents used in tooth whitening are hydrogen peroxide, carbamide peroxide and sodium perborate (Teles Júnior et al., 2018; Riehl et al., 2008).

Tooth whitening procedures may be implemented using at-home or in-office techniques. Home whitening processes make use of active agents in low concentrations and, through a customized teeth whitening tray, exposes the patient's dental structures to treatment for several hours. This procedure may be performed by the patient themselves. Conversely, during in-office teeth whitening, one of the strategies most commonly used by dental surgeons, whitening agents are applied in high concentrations for several minutes. This procedure must therefore be performed in a dental office (Kielbassa et al., 2015; Rezende et al., 2016).

In-office teeth whitening may involve more than one application of whitening gel on dental structures and usually comprises two or three stages (Greenwall et al., 2001). Although products that involve single-step applications may reduce the procedure's clinical period, adjustments must be made to their formulations to ensure that the uninterrupted application time does not harm dental structures (Cavalli et al., 2019).

The human tooth, as well as the structures that comprise it, may be permeated by certain substances. Dentin tubules allows certain fluids to reach the innermost part of the tooth—the pulp (Kwon et al., 2012). Gels used for tooth whitening are formed or give rise to hydrogen peroxide. This compound is able to not only promote teeth whitening by interacting with the molecules that color teeth and, through the release of free radicals, facilitate their removal, but also traverses dental structures and, through means of dentin tubules, reach the tooth pulp. However, prolonged contact with hydrogen peroxide-based gels may cause large amounts of this agent to reach the tooth pulp, which leads to a condition known as dental hypersensitivity (Epple; Meyer; ENAX, 2019).

Dental whitening performed in-office technique involves the use of agents, such as hydrogen peroxide, in high concentrations that provide remarkable whitening results in one session. However, due to the high concentrations of these agents that are applied, large amounts of their active ingredients can reach the innermost layers of the tooth, particularly the tooth pulp, and cause sensory and cellular changes. The main adverse effect of this type of treatment, therefore, is dental hypersensitivity, which is associated with the amount of free radicals that traverse the dentin tubules and reach the tooth pulp (Costa et al., 2010).

### PRIOR ART

During in-office teeth whitening, high concentration hydrogen peroxide gels are applied in sessions that last between 20 and 50 minutes. However, since the procedure causes dental sensitivity in a high percentage of patients (Kielbassa et al., 2015; Rezende et al., 2016), several clinical studies have been carried out to attempt to improve application protocols or, in other words, reduce adverse effects by maintaining whitening patterns.

Reis et al. (2011), for example, evaluated in a randomized clinical trial whether the use of an acidic hydrogen peroxide gel in in-office whitening applied for 1×45 minutes would present the same whitening rate and levels of tooth sensitivity when compared to 3×15-minute applications. In-office bleaching was performed using 35% hydrogen peroxide gel.

In one group, the whitening agent was updated every 15 minutes, on three occasions during each whitening visit. In the other study group, the gel was left unchanged on teeth's buccal surfaces for 45 minutes during each whitening session. This protocol was repeated after one week. A bleaching evaluation was performed using a value-oriented color scale after each whitening session.

Patients recorded dental sensitivity on a scale of 0-4. The results obtained showed that the use of the gel for a single period of 45 minutes decreased the effectiveness of whitening (p<0.05) and increased the intensity of tooth sensitivity (p=0.04) when compared to the 3×15 minute group. The authors of the study concluded that the application of a 35% hydrogen peroxide gel in in-office bleaching should preferably be performed during three 15-minute applications.

Kossatz et al. (2012) evaluated in randomized clinical trial the tooth sensitivity and the whitening efficacy associated with the use of bleaching gels in the office, one of which was applied for 3×15 minutes (low pH) and the other for 40 min (alkaline pH) without interruption. Two whitening sessions were performed with a one-week interval between them. The authors recorded color subjectively and dental sensitivity on a scale from 0 to 4. The two groups present similar and significant whitening effects (p>0.05). However, the study group in which hydrogen peroxide was not exchanged (1×40 min) presented a significantly lower pattern of tooth sensitivity (risk and intensity) when compared to the 3×15 min whitening regimen (p<0.05). As a result, the authors of the study recommended that in-office bleaching not be exchanged.

Kose et al. (2016) evaluated in a randomized clinical trial the effectiveness of bleaching and the tooth sensitivity in in-office bleaching in which acidic hydrogen peroxide was applied under different protocols. Patients were randomly assigned to three groups: hydrogen peroxide was applied to the buccal surface of the upper anterior teeth in one (1×15), two (2×15), or three (3×15) 15-minute applications. Two bleaching sessions were performed with an interval of one week between each session. Teeth color was evaluated using a visual color guide and spectrophotometer before and 30 days after bleaching. Participants also measured dental sensitivity on a numerical scale.

All groups presented some degree of whitening. Lower whitening levels were only observed at 1×15 (p<0.05). However, the absolute risk of tooth sensitivity was also lower for the 1×15 group when compared to the remaining groups (p<0.05). The dental sensitivity intensity in the 3×15 group was statistically higher than that associated with the remaining protocols (p<0.05). It was concluded that a single 15-minute application produced a lower level of tooth sensitivity but reduced whitening effect. Conversely, the 2×15 whitening protocol produced an excellent whitening pattern, but presented reduced tooth sensitivity intensity compared to the 3×15 group.

Loguercio et al. (2017) evaluated in a randomized 5 clinical trial the tooth sensitivity and whitening efficacy of two hydrogen peroxide gels with different pH levels—one acidic and the other neutral—in in-office bleaching. Teeth were whitened during two sessions, with an interval of 1 week between sessions. Each session comprised three applications of 8 min each, according to the manufacturer's instructions. Changes in color were assessed using subjective and objective methods. Participants assessed dental sensitivity using a visual analog scale.

Both the group treated with neutral pH whitening gel and the group treated with acidic pH whitening gel presented the same level of whitening after 30 days of clinical evaluation (p>0.05). However, the use of an in-office neutral bleaching gel significantly reduced the absolute risk and the intensity of tooth sensitivity when compared to the acid bleaching gel (p<0.05). The use of an in-office bleaching agent with a neutral pH produced the same degree of whitening as an acid bleaching gel, but with a reduced risk and intensity of dental sensitivity.

Martins et al. 2018 evaluated in a randomized clinical trial the whitening efficacy and tooth sensitivity of a 38% alkaline hydrogen peroxide whitening agent used in in-office whitening, administered in single 40-minute or two 20-minute applications. Teeth were whitened during two sessions, with an interval of 1 week between sessions under a split-mouth design.

Changes in color were assessed using subjective and objective methods at baseline and 30 days after the second session. Tooth sensitivity was recorded within 48 hours using a visual analog scale. Whitening pattern and number of patients presenting similar tooth sensitivity were observed in both groups after 30 days of clinical evaluation (p>0.05). Researchers concluded that the application of an in-office bleaching agent for 40 minutes produced the same degree of tooth whitening and sensitivity as two 20-minute bleaching agent applications. The former method must preferably be applied given that a 40-minute application is simpler.

Meireles et al. (2021) evaluated, during the randomized clinical trial described herein, efficacy and dental sensitivity in a 37.5% alkaline hydrogen peroxide in-office or whitening procedure applied for two three 8-minute applications during each clinical session. Three clinical sessions were performed with an interval of 1 week between sessions. Color was assessed using a digital spectrophotometer. Tooth sensitivity was measured during and within 48 hours of whitening using a five-point numerical rating scale. Both groups presented significant tooth whitening 1 week after whitening sessions (p<0.05). Additionally, there was no difference between the groups in terms of absolute high risk and intensity of sensitivity (p>0.05). In-office whitening comprising two applications of hydrogen peroxide is recommended, as it reduces application time and maintains the degree of whitening, risk and intensity of dental sensitivity when compared to the use of three hydrogen peroxide applications.

Based on an analysis of the clinical studies described so far, it is clear that results regarding the need for exchange and the application period for commercially available in-office whitening gels are a point of controversy. Additionally, whitening efficacy may also vary according to whitening gels' pH levels. A known means of increase bleaching efficacy, for example, involves increasing the hydrogen peroxide concentration and/or composition pH. However, peroxide concentration is associated, along with acidic pHs, with the induction of undesired dental sensitivity after whitening. Additionally, increasing the composition pH, while improving bleaching efficacy, reduces r the stability of the hydrogen peroxide present in the solution, thereby reducing the composition's shelf life. Such dilemmas have been address in the prior art, but have not resulted in a composition such as that described herein.

For example, U.S. Pat. No. 6,488,914B2 and U.S. Pat. No. 5,922,307B2 describe an at-home tooth whitening compositions formulated at a pH of between 6.0 and 10.0 and that contain a thickening agent, hydrogen peroxide stabilizer, buffer, and calcium chelator. Despite including sodium stannate among the available hydrogen peroxide stabilizers, compositions present significantly lower concentrations of the active agent. Furthermore, the compositions provided in U.S. Pat. No. 6,488,914B2 and U.S. Pat. No. 5,922,307B2 offer whitening levels (as measured under the CIELAB protocol) between 2 and 4 times lower than the present invention.

Additionally, application US2004/0101497A1 describes two-component tooth whitening compositions. The formulation's active component may contain hydrogen peroxide (35%) as a bleaching agent at 17.14% of the composition, in addition to thickening agents, buffers, chelators and stabilizers. Sodium stannate is included among available hydrogen peroxide stabilizers. It is important to reiterate that the present invention's composition provides an improved level of whitening over a shorter period of time (30 versus 60 minutes).

The present invention's bleaching composition contains hydrogen peroxide in a useful concentration of 35% and formulated at a pH close to neutral. The presence of hydrogen peroxide stabilizers attenuates the observed drop in the composition's pH during application. As a result, the composition described herein provides levels of whitening equivalent to that provided by compositions under the prior art, but in a single application and with a reduced level of penetration of hydrogen peroxide in the tooth pulp, thereby reducing the unwanted sensitivity common in this type of procedure (Silva et al, in press, 2022).

### SUMMARY OF THE INVENTION

This invention comprises a hydrogen peroxide-based dental whitening composition with an active component comprising at a minimum 35% (v/v) hydrogen peroxide, sodium stannate, phosphoric acid and ethylenediaminetetraacetic acid (EDTA). It also includes a thickening component that comprises an acrylic resin and water.

In a particular embodiment, the invention's dental whitening composition presents a sodium stannate concentration of 0.63% of the total volume (v/v). In another form of the invention, the dental whitening composition presents a pH between 6.5 and 7.5.

An additional aspect of the invention encompasses a kit comprising a syringe with the active component of the dental whitening composition and thickening component, respectively. In specific embodiments, the kit further comprises a neutralizing component and a gingival protector. In certain aspects, the kit may further include instructions for preparing the dental whitening composition by mixing active and thickening components.

Yet another aspect of the invention provides a tooth whitening method for exclusively aesthetic purposes, in which a tooth whitening composition is applied to patient's teeth.

Under specific embodiments of the invention, the whitening method involves applying one or more stages of the bleaching composition. The preferred method comprises a stage in which the bleaching composition is applied for 30 minutes.

### BRIEF DESCRIPTION OF DRAWINGS

FIG. 1 presents an illustration of the CIELAB color space.

FIG. 2 illustrates the whitening efficacy of the invention's composition compared to the whitening gel prepared by Balladares et al (2019). Each group received the respective treatment during the course of 3 independent sessions, with an interval of one week between each session, in order to simulate office visits. Levels of whitening achieved with under each protocol at weeks 1, 2, and 3 are depicted in tables A, B, and C, respectively.

FIG. 3 illustrates the methodology used to evaluate the level of penetration of hydrogen peroxide into the pulp of the patients' dental elements. A methodology adapted from Favoreto et al., 2020.

FIG. 4 presents results of a study into the penetration of hydrogen peroxide into the pulp of the patients' dental elements in μg/mL.

FIG. 5 presents pH measurements for the invention's tooth whitening composition under the different protocols tested.

FIG. 6 demonstrates pH values after different periods of exposure times to the invention's bleaching gel compared to the bleaching gel used under the prior art (values by Balladares et al., 2019).

### DETAILED DESCRIPTION OF THE INVENTION

Before describing the invention, it is necessary to emphasize that the terms used throughout these specifications are assigned standard meanings under the prior art within the context of disclosure, and in the specific context in which each term is used, unless otherwise specified. Certain terms that are used to describe disclosure are discussed below, or in other sections of the specifications in order to provide practitioners with additional guidance regarding descriptions of the disclosure. The publications cited herein and the materials for which they are cited are specifically incorporated in their entirety by reference.

It must be understood that the same idea may be express in several different manners. Accordingly, alternative language and synonyms may be used for any one or more of the terms discussed in this work. Special meaning must not be assigned with regards to whether a term is elaborated or discussed herein. Synonyms for certain terms are provided; however, exemplification of certain synonyms does not exclude the potential use of other terms that are not listed herein.

The present invention provides a tooth whitening composition containing hydrogen peroxide in a final concentration of at a minimum 35% of the composition (v/v) by total volume, one or more hydrogen peroxide stabilizers, and a minimum of one acidifier and thickening agent, respectively.

It is recognized that a technical dilemma exists with regards to the stability of hydrogen peroxide and safety in tooth whitening compositions. On the one hand, hydrogen peroxide presents greater stability under more acidic pH ranges. On the other hand, the more acidic the whitening composition, the greater the extent of penetration of the peroxide into the deepest layers of the tooth, which is correlated with an undesirable level of sensitivity after whitening. For this reason, the invention's composition contains a combination of stabilizers and acidifiers that allow for a formulation and application in ranges close to neutral pH, which in turn resolves the above-mentioned technical dilemma.

There are three available classes of hydrogen peroxide stabilizers. Certain stabilizers, such as ethylenediaminetetraacetic acid (EDTA), act as metal chelators since metal ions can react with hydrogen peroxide in the capacity of reductants and thereby activate peroxide decomposition. Another class of stabilizers consist of antioxidants such as sodium sulfite. The third type of stabilizer can specifically stabilize hydrogen peroxide and comprises, for example, tin salts, alkali stannates (e.g. sodium stannate), phosphates, pyrophosphates, phosphoric acid, and similar materials.

Thickening agents must be used to obtain a viscous gel that can adhere to the surface of the teeth during whitening. Due to hydrogen peroxide's high oxidizing capacity, the thickening agent used must be stable or inert in relation to hydrogen peroxide within the bleaching composition. Given their stability at an acidic pH, thickening agents must be stable or at a minimum effective at acidic pH. In the context of the present invention, thickening agents may be selected from, but acrylic resin, poly(ethylene oxide), are not limited to, polyvinylpyrrolidone (PVP), a polyethylene oxide and polypropylene oxide copolymer, and corresponding mixtures.

In a preferred form of the invention, the tooth whitening composition described herein comprises 35% (v/v) hydrogen peroxide, sodium stannate, EDTA, phosphoric acid, and an acrylic resin. Under even more preferred embodiments of the invention, sodium stannate corresponds to 0.63% of the composition, and EDTA, phosphoric acid, and acrylic resin to 0.1%, 0.5% and 7.0%, respectively.

The invention's bleaching composition may be prepared by mixing two parts—an active component and a thickening agent—during application. In this context, the active component, responsible for the invention's whitening effect, comprises a bleaching agent—hydrogen peroxide-one or more hydrogen peroxide stabilizers and at a minimum one acidifier. The composition's thickening agent component, as expected, comprises a thickening agent, e.g., acrylic resin, dispersed in purified water.

An auxiliary embodiment of the invention therefore comprises a kit that includes at a minimum the active component and the bleaching composition's thickening component. Additional embodiments of the kit may comprise other accessory elements known to skilled professionals, such as, for example, instructions for preparing the bleaching composition by mixing components immediately prior to use, as well as applying it during in-office bleaching treatments.

In one specific form, the kit comprises a minimum of one vial or syringe containing the active component (hydrogen peroxide) and the thickening agent component, respectively, and application accessories such as, for example, applicator tips and connectors, as well as instructions for use.

In an even more particular embodiment, the kit comprises a minimum of one vial or syringe with at a minimum approximately 0.92 g of the active component (hydrogen peroxide), a vial or syringe with approximately 0.30 g of the thickening agent component, application accessories and instructions for use.

The composition described herein has an unprecedented whitening efficacy equivalent to those already available; however, it is more efficient and safer. In the context of the present invention, ‘whitening efficacy’ denotes the degree of whitening provided by the composition through a specific treatment protocol as measured using a specific standard scale. Accordingly, the expression ‘whitening efficiency’ denotes the period of time and number of sessions required for the composition to provide the desired whitening effect.

The effectiveness and efficiency of tooth whitening processes can be measured in different manners depending on the specificity of desired evaluation. In clinical comparisons, where a distinction is made between tones in the tooth's initial state—prior to whitening—and the final state—after whitening—in order to visually highlight whitening, comparative methods comprising dental elements subject to visual scales, such as the Vita Scale, for example, may be used (Thanathornwong; Suebnukarn; Quivirach, 2015).

During quantitative evaluations used in studies and research, devices that detect the passage of light through dental elements—called spectrophotometers—are used to provide more accurate values. This colorimetric measurement is based on the fact that colors, for evaluation purposes, present three distinct dimensions: hue, chroma and value. Hue determines the color itself, for example, red, yellow, or blue. Chroma determines color intensity, which is also known as saturation or degree of saturation. Value, in turn, is related to the brightness and reflection of light on patients' teeth. Together, devices that measure tooth color use these parameters to provide more accurate values than visual comparisons provide, thereby avoiding subjective evaluations. Individual evaluation of these parameters allows for a more precise identification of the influence whitening treatments have on tooth color (Teles Junior et al., 2018; Thanathornwong; Suebnukarn; Quivirach, 2015).

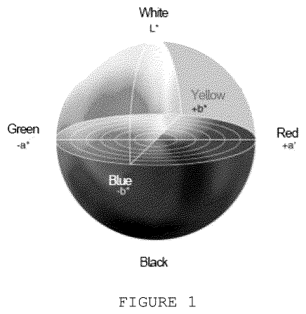

Quantitative evaluations made using spectrophotometers are commonly identified through CIE (International Commission on Illumination) parameters through the LAB colorimetric space (or L*a*b*), in which the L* coordinate corresponds to brightness (value 0 corresponds to darker tones, and the value 100 to lighter tones), the a* value to colorimetric variation between red (+60) and green (−60), and the b* value to the variation between yellow (+60) and blue (−60). This system is also known as CIELAB (Wheatherall; Coombs, 1992) (FIG. 1).

However, since the color of human teeth generally falls within a small spectrum of this color space, an adjustment index may be implemented under CIELAB to ensure it more closely reflects realities in the dental industry. Such an adjustment index can be observed, for example, with the whiteness index for dentistry (WID), a commonly used adjustment factor for dental whitening assessments. Under the index, variations in yellowish levels are key factors in determining the color space value, in which low yellow values are associated with higher levels of whiteness, while higher values correspond to more yellowish teeth (Luo et al., 2009).

In a complementary manner, within the context of this invention, the safety of a dental whitening composition refers to a series of properties related to the intensity of unwanted adverse effects of in-office dental whitening treatment using hydrogen peroxide-based solution.

Hydrogen peroxide, when reacting with dental structures, gives rise to molecules with low molecular weight-reactive oxygen species, which are able to traverse hard dental tissues (Eimar et al., 2012) and damage tooth pulp cells (Soares et al., 2014). Although the mechanisms behind the adverse effects caused by hydrogen peroxide-based whitening gels have not yet been completely determined, it is believed that the observed cell damage is related to these effects, which include dental sensitivity. Identified evidence includes increased cell damage in cases involving greater penetration of hydrogen peroxide into tooth pulp (Soares et al., 2014).

It is therefore possible to evaluate the safety of teeth whiteners containing hydrogen peroxide through methodologies that quantify the level of hydrogen peroxide that reaches teeth's pulp after a treatment. For example, hydrogen peroxide taken from a tooth pulp sample, if exposed to leukocrystal violet dye, acquires staining proportional to its concentration that can be quantified using a spectrophotometer.

Given the efficacy, efficiency and safety of the invention's whitening composition, a method of tooth whitening is also provided for exclusively aesthetic purposes that is useful in dental offices, comprising the application of the whitening composition described herein to patients' teeth. This method consists of applying the composition in one or more sessions, which may vary in length, such as, for example, approximately 10 minutes, 20 minutes, 30 minutes, 40 minutes, 50 minutes, or 60 minutes. This method preferably involves applying the invention's bleaching composition in a single session lasting 30 minutes.

The following sections illustrates the invention through examples of embodiments that do not exhaust the possibilities of the inventive concept described herein, but represent the entirety of potential manifestations. The following examples are presented to provide professionals with a complete description of how the tooth whitening compositions of this invention are prepared, evaluated and employed. A professional skilled in this field, given the present disclosure, will recognize that many changes can be made to specific embodiments that are disclosed and a similar or equivalent result obtained without moving away from the spirit and scope of the invention.

### EXAMPLES

### Example 1—Preparation of the Dental Whitening Composition

The invention's bleaching composition was prepared in two parts. The first part, responsible for the desired action, is composed of the active agent-hydrogen peroxide (hydrogen peroxide 50% in water, with a final concentration of 35%), preservative—EDTA—, stabilizer—sodium stannate—and an additive-phosphoric acid 85% (table 1).

TABLE 1
Invention's dental whitening composition - phase 1.

| Component | % | Function |
|---|---|---|
| Hydrogen peroxide 50% | 91.77 | Whitening agent |
| EDTA | 0.1 | Preservative |
| Sodium Stannate | 0.63 | Stabilizer |
| Phosphoric Acid 85% | 0.5 | Acidifier / |
| | | Stabilizer |

The second part of the bleaching composition serves a thickening function and is composed of acrylic resin that is conveyed in water (Table 2).

TABLE 2
Invention's dental whitening composition - phase 2.

| Component | % | Function |
|---|---|---|
| Acrylic resin | 7.0 | Thickening agent |

### Example 2—pH Kinetic Evaluation of Bleaching Composition During Dental Application

The pH was assessed using a standard laboratory pH-meter (ExStick pH100, Extech). The invention's bleaching composition was applied to the buccal surface of bovine dental elements and triplicate results provided for each group were analyzed. Mean values for each group were considered. As a result, the bleaching gel's pH was measured at 4 different times: immediately before the product coming into contact with the patients' teeth (to) and after 20 minutes (t₂₀), 30 minutes (t₃₀), and 40 minutes of the gel coming into contact with the patients' teeth (t₄₀).

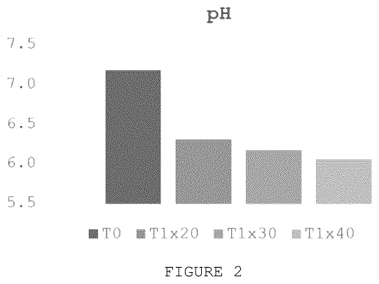

The results of pH evaluations (FIG. 2) showed that, at tor, the invention's bleaching gel has a slightly alkaline pH (7.16) near neutrality. For the remaining groups, the pH values were very close to one another during the different periods analyzed: pH at t₂₀=6.30; pH at t₃₀=6.16; and pH at t₄₀=6.06. During a statistical evaluation between groups using the Tukey test, it was observed that pH values for experimental groups (t₂₀, t₃₀ and t₄₀) differed statistically for initial pH value (to). A statistical difference was observed for the pH values between t₂₀ and two between experimental groups. There was no statistically significant difference observed between remaining experimental groups during the comparison.

When comparing the pH values of the invention's dental whitening composition with the values obtained using the whitening composition under the existing prior art (table 3), as described in Balladares et al. (2019), it was observed that the pH value at to is more acidic for under the prior art formulation (6.4-6.3) than for the composition described herein (7.16). Additionally, the composition prepared by Balladares et al. (2019) also presents a higher level of acidity (5.6) than the invention's composition at t₃₀ (6.16) or during longer exposure periods, such as t₄₀ (6.06). It was also observed that the invention's pH at t₄₀ presented lower acidity values than those of the three-exposure period analyzed in the study by Balladares et al. (2019). The composition of the whitener used by Balladares et al. (2019) is presented in Table 4.

TABLE 3
pH values during different periods of exposure to the
bleaching gel prepared by Balladares et al. (2019).

| Application period | Average pH values |
|---|---|
| Before application | 6.4-6.3* |
| 15 minutes | 5.8 |
| 30 minutes | 5.6 |
| 45 minutes | 5.2 |

*The authors of the study provided two average pH values for the initial period.

TABLE 4
Composition of the whitener prepared by Balladares et al. (2019)

| Component | % | Function |
|---|---|---|
| Hydrogen peroxide 35% | 39.8 | Whitening agent |
| Hydrogen peroxide 60% | 49.9 | Whitening agent |
| EDTA | 0.1 | Preservative |
| Acrylic resin | 10.2 | Thickening agent |

As a result, the results of the study into the bleaching gel's pH as part of the invention presented a basic pH when mixing hydrogen peroxide with the thickening agent. When evaluated after contact with dental elements, the decrease in pH reached slightly acidic values during all evaluated periods. More acidic pH values are associated with greater changes in dental structure (Sa et al., 2012; Magalhães et al., 2011). These changes, in turn, cause adverse effects, such as dental hypersensitivity, which also presents a higher incidence and greater intensity when low pH in-office whitening gels are used (Loguercio et al., 2017).

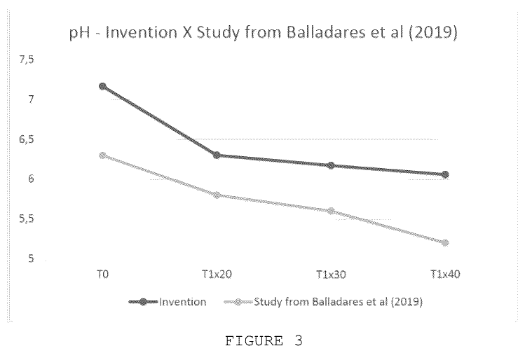

During comparison between the invention's bleaching composition and an example of the prior art (FIG. 3), it was observed that the invention presents a more alkaline pH. This was observed through an initial assessment of the bleaching gel's pH when mixing the hydrogen peroxide with the thickening agent, as well as during proposed exposure times. When only products' pH was exclusively analyzed during contact with the dental structures, the invention's composition presented a higher pH stability (ranging from 6.30 at t₂₀ to 6.06 at t₄₀) than the bleaching gel under the prior art (ranging from 5.8 at t₁₅ to 5.2 at t₄₅).

The formulation used in the present invention's whitening composition presents two main advantages in relation to the formulation prepared by Balladares et al. (2019): sodium stannate and phosphoric acid. Results suggest that these components, at the proportions used, provide the surprising stability of the bleaching composition described herein when compared to the prior art.

### Example 3—Whitening Efficacy Assessment

To verify the invention's whitening efficacy, tests that compared different whitening protocols using coffee-stained bovine teeth as a model were performed. Teeth were whitened according to treatment periods previously established for each group (table 3).

The invention's composition was tested during 2×20-minute applications, or 1×30-minute or 1×40-minute application. An experimental group was dedicated to the whitener used under the prior art (Balladares et al., 2019), evaluated under a protocol consisting of 2 applications of 20 minutes each for comparative purposes.

An Easyshade® spectrophotometer was used for colorimetric evaluation. The CIALAB WID color space system was selected for the purposes of quantitative comparisons between the level of whiteness among dental elements. Three whitening sessions were performed for each group, and 4 whiteness readings were taken: one before the whitening intervention and three after each whitening session. Data were statistically evaluated through a mean comparison test (Tukey's test) while considering a 95% confidence interval.

TABLE 3
Whitening protocols by group

| Identification | Group | Protocol |
|---|---|---|
| White | | |
| (Not stained in coffee and not | G1 | — |
| bleached) | | |
| Negative control | | |
| (Stained in coffee and without | G2 | — |
| undergoing whitening) | | |
| Comparative whitening gel (yellow | G3 | 2 × 20 min. |
| dye) | | |
| Invention's bleaching gel (red dye) | G4 | 2 × 20 min. |
| Invention's bleaching gel (red dye) | G5 | 1 × 30 min. |
| Invention's bleaching gel (red dye) | G6 | 1 × 40 min. |

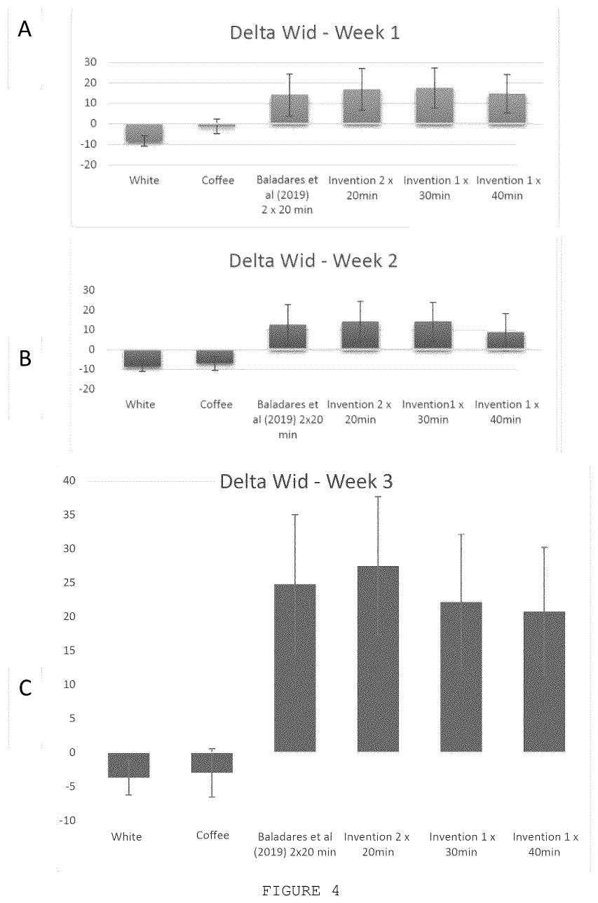

The results of this study showed that all protocols evaluated effectively whitened the bovine dental elements. Additionally, it was also observed that the degree of whitening between groups was comparable and did not present statistically significant difference (FIG. 4).

As a result, due to the practicality of the protocol of one application, which results in a shorter clinical procedure period, a single application protocol was considered the most suitable for the invention's bleaching gel. Following this same logic, the protocol comprising a shorter application period—1×30-minutes application—was found to be preferable since it provided the same degree of whitening as the remaining product applications studied.

### Example 4—Safety Assessment

The safety of the invention's bleaching composition was evaluated through hydrogen peroxide penetration tests performed on human tooth pulp. Different protocols for exposing the teeth to the composition were implemented, and the number of applications and the period the gel remained into contact with the teeth varied. Four experimental groups were therefore formed, namely: one 30-minute application (1×30 min), two 15-minute applications (2×15 min), one 40-minute application (1×40 min); two 20-minute applications (2×20 min).

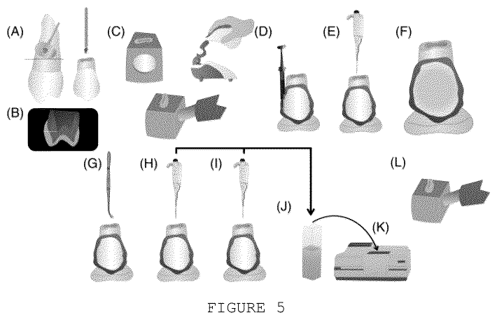

In order to evaluate the permeation of hydrogen peroxide at the dental pulp, dental elements were initially cut in half to access the pulp chamber (FIG. 5A). The thickness of the specimens was then evaluated, and radiography of the elements performed (FIG. 5B). In order to standardize the position of the spectrophotometer, a impression made from a dense addition silicone paste (Futura AD, DFL) was prepared. The initial color of the elements was determined using a spectrophotometer (Extech pH 100: ExStik pH Meter; Extech instruments, Nashua, New Hampshire; FIG. 5C).

Specimens were isolated on their labial face using a light-polymerized resin barrier (Top dam, FGM, Joinville, SC, Brazil) (FIG. 5D). It was applied 25 μL of acetate buffer (Leucocrystal Violet, Sigma Chemical Co., St Louis, Missouri) to each element's pulp chamber (FIG. 5E). Teeth were subject to the whitening procedure using the invention's composition according to their group protocol (FIG. 5F).

After the bleaching stage, the bleaching gel was removed using cotton swabs and tweezers (FIG. 5G), and the acetate buffer solution was removed from inside the pulp cavity using a micropipette (FIG. 5H). The cavity was cleaned using 25 μL of buffer an additional 4 times (FIG. 5I), and the 5 buffer solutions used to remove the cavity were mixed with the reagent solution (100 μL of 0.5 mg/mL violet leukocrystal, 50 μL of horseradish peroxidase enzyme—Peroxidase Type VI-A, Sigma Chemical Co., St. Louis, Missouri—at 1 mg/mL and 2,725 μL ultrafiltered water) for subsequent evaluation using spectrophotometer (Varian, Palo Alto, California; reading at 596 nm).

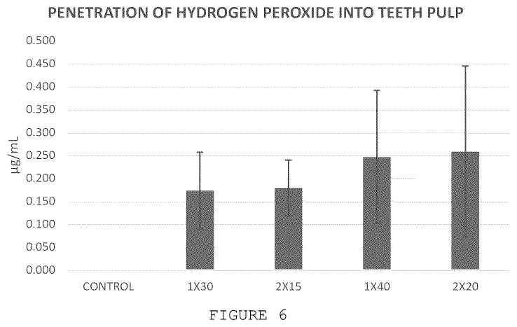

The results of this experiment (FIG. 6) demonstrated that the single application protocol for the invention's bleaching composition, which totaled 30 minutes, presented the lowest amount of hydrogen peroxide penetration in the pulp cavity (0.173 μg/mL). In contrast, the group corresponding to the protocol comprising two 20-minute applications each presented the highest amounts of hydrogen peroxide in the dental pulp (0.258 μg/mL).

As a result, based on the results of the study presented, it was determined that 30-minute applications of the invention's bleaching gel provided the lowest level of penetration of hydrogen peroxide into the pulp of the teeth studied. Due to its practicality, this single application protocol, which reduces total clinical time in relation to the two application protocol, together with the reduction in permeation of the whitening agent in the dental pulp, was considered the most appropriate.

### BIBLIOGRAPHIC REFERENCES

BALLADARES, Luis et al. Effects of pH and application technique of in-office bleaching gels on hydrogen peroxide penetration into the pulp chamber. Operative dentistry, v. 44, n. 6, p. 659-667, 2019.
CAVALLI, Vanessa et al. Decomposition rate, pH, and enamel color alteration of at-home and in-office bleaching agents. Brazilian Dental Journal, v. 30, p. 385-396, 2019.
COSTA, Carlos Alberto de Souza et al. Human pulp responses to in-office tooth bleaching. Oral Surgery, Oral Medicine, Oral Pathology, Oral Radiology, and Endodontology, v. 109, n. 4, p. e59-e64, 2010.
EIMAR, Hazem et al. Hydrogen peroxide whitens teeth by oxidizing the organic structure. Journal of Dentistry, v. 40, p. e25-e33, 2012.
EPPLE, Matthias; MEYER, Frederic; ENAX, Joachim. A critical review of modern concepts for teeth whitening. Dentistry journal, v. 7, n. 3, p. 79, 2019.
FAVORETO, Michael W. et al. A novel carbamide peroxide polymeric nanoparticle bleaching gel: Color change and hydrogen peroxide penetration inside the pulp cavity. Journal of Esthetic and Restorative Dentistry, v. 33, n. 2, p. 277-283, 2021.
FREIRE, Andrea et al. Reaction kinetics of sodium ascorbate and dental bleaching gel. journal of dentistry, v. 37, n. 12, p. 932-936, 2009.
KWON, So Ran et al. Dynamic model of hydrogen peroxide diffusion kinetics into the pulp cavity. J Contemp Dent Pract, v. 13, n. 4, p. 440-5, 2012.
LOGUERCIO, A. D. et al. Effect of acidity of in-office bleaching gels on tooth sensitivity and whitening: a two-center double-blind randomized clinical trial. Clinical oral investigations, v. 21, n. 9, p. 2811-2818, 2017.
LUO, Wen et al. Development of a whiteness index for dentistry. Journal of Dentistry, v. 37, p. e21-e26, 2009.
MAGALHÃES, Juliana G. et al. Microhardness change of enamel due to bleaching with in-office bleaching gels of different acidity. Acta Odontologica Scandinavica, v. 70, n. 2, p. 122-126, 2012.
RIEHL, H. et al. Whitening of vital and non-vital teeth: a critical perspective. FONSECA, AS Aesthetic Dentistry. The art of perfection. São Paulo: Artes Médicas, p. 499-565, 2008.
SA, Yue et al. Effects of two in-office bleaching agents with different pH values on enamel surface structure and color: an in situ vs. in vitro study. Journal of dentistry, v. 40, p. e26-e34, 2012.
SOARES, Diana Gabriela et al. Concentrations of and application protocols for hydrogen peroxide bleaching gels: effects on pulp cell viability and whitening efficacy. Journal of dentistry, v. 42, n. 2, p. 185-198, 2014.
TELES JUNIOR, Marcelo et al. Dental bleaching and new possibilities: literature review. Health Science Journal, v. 12, n. 6, p. 0-0, 2018.
THANATHORNWONG, Bhornsawan; SUEBNUKARN, Siriwan; OUIVIRACH, Kan. Decision support system for predicting color change after tooth whitening. Computer methods and programs in biomedicine, v. 125, p. 88-93, 2016.
WEATHERALL, Ian L.; COOMBS, Bernard D. Skin color measurements in terms of CIELAB color space values. Journal of investigative dermatology, v. 99, n. 4, p. 468-473, 1992.

1. A hydrogen peroxide based dental whitening composition comprising:
an active component, which includes:
at a minimum 35% (v/v) hydrogen peroxide;
sodium stannate;
phosphoric acid;
ethylenediaminetetraacetic acid (EDTA); and
a thickening component, comprising:
an acrylic resin; and
water.

2. The hydrogen peroxide based dental whitening composition according to claim 1, wherein a concentration of sodium stannate is 0.63% (v/v).

3. The hydrogen peroxide based dental whitening composition according to claim 1, wherein a pH totals between 6.5 and 7.5.

4. A kit comprising at least one vial or syringe containing the active component according to claim 1 and one vial or syringe containing the thickening component as defined in claim 1.

5. The kit according to claim 4, wherein the vial or syringe containing the active component contains at a minimum approximately 0.92 g of the active component and a vial or syringe contains 0.30 g of the thickening agent.

6. The kit according to claim 4, further comprising instructions for preparing the tooth whitening composition by mixing active and thickening components.

7. A tooth whitening method comprising applying the dental whitening composition according to claim 1 to a patient's tooth.

8. The method according to claim 7, consisting of one or more steps of applying the composition.

9. The method according to claim 8, wherein each application step may endure for between 20 and 40 minutes.

10. The method according to claim 8, consisting of a single application stage lasting 30 minutes.
